(19) United States
(12) Patent Application Publication
(10) Pub. No.: US 2010/0174551 A1
(43) Pub. Date: Jul. 8, 2010

(54) SYSTEMS AND METHODS OF REGISTERING FOR EMERGENCY MEDICAL SERVICES

(76) Inventor: Tyler Kiley, Powder Springs, GA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/602,190

(22) PCT Filed: May 30, 2008
(86) PCT No.: PCT/US08/65353

§ 371 Date: Nov. 30, 2009

Related U.S. Application Data
(60) Provisional application No. 60/941,139, filed on May 31, 2007.

(51) Int. Cl. G06Q 10/00; G06Q 90/00

(52) U.S. Cl. 705/2; 705/500; 705/5; 726/5

(57) ABSTRACT

Various systems and methods are provided for registering for emergency medical services. In one embodiment, a method includes providing a current wait time for an emergency medical facility; receiving a request to reserve a visitation time for the emergency medical facility, the visitation time after the current wait time; requesting patient information; and notifying the emergency medical facility of the reserved visitation time.

### CROSS REFERENCE TO RELATED APPLICATIONS

This application claims priority to copending U.S. provisional application entitled “SYSTEMS AND METHODS OF REGISTERING FOR EMERGENCY MEDICAL SERVICES” having Ser. No. 60/941,139, filed May 31, 2007, which is entirely incorporated herein by reference.

### BACKGROUND

Everyone, at one time or another, has a need for emergency medical attention. Generally, emergency rooms and urgent care facilities address a patient's needs in order of arrival and severity of injury. Depending on the capacity of the hospital or urgent care facility, patients may experience extended waiting times before seeing a physician or receiving treatment.

### SUMMARY

Embodiments of the present disclosure are related to systems and methods related to registering for emergency medical services.

Briefly described, one embodiment, among others, comprises a method. The method comprises providing a current wait time for an emergency medical facility; receiving a request to reserve a visitation time for the emergency medical facility, the visitation time after the current wait time; requesting patient information; and notifying the emergency medical facility of the reserved visitation time.

Another embodiment, among others, comprises a method. The method comprises receiving a request to view a virtual waiting room associated with an emergency medical facility; and providing the virtual waiting room including a list of patients who have a reserved visitation time for the emergency medical facility.

Another embodiment, among others, comprises a method. The method comprises receiving a username and a corresponding password; determining an emergency medical facility associated with the username; receiving an estimated waiting time; updating a current waiting time associated with the emergency medical facility; and providing notification of the updated current waiting time to a patient having a reserved visitation time for the emergency medical facility.

Other systems, methods, features, and advantages of the present disclosure will be or become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 is an illustration of a triage nurse waiting time entry form in accordance with one embodiment of the present disclosure;

FIG. 2 is an illustration of a single hospital waiting time screen in accordance with one embodiment of the present disclosure;

FIGS. 3A and 3B are illustrations of a patient information form in accordance with one embodiment of the present disclosure;

FIGS. 4A and 4B are illustrations of patient confirmation screens in accordance with one embodiment of the present disclosure;

FIG. 5 is an illustration of a patient medical history form in accordance with one embodiment of the present disclosure;

FIG. 6 is a flow chart illustrating a user process for registering for emergency medical services using the forms of FIGS. 3-5 in accordance with one embodiment of the present disclosure;

FIG. 7 is a flow chart illustrating a server process for registering for emergency medical services as illustrated in FIG. 6 in accordance with one embodiment of the present disclosure;

FIG. 8 is an illustration of an emergency medical facility login screen in accordance with one embodiment of the present disclosure;

FIG. 9 is an illustration of a facility home page including the triage nurse waiting time entry form information of FIG. 1 in accordance with one embodiment of the present disclosure;

FIG. 10 is an illustration of a virtual waiting room display which may be accessed from the facility home page of FIG. 9 in accordance with one embodiment of the present disclosure;

FIG. 11 is an illustration of a patient information summary which may be viewed or printed from the virtual waiting room of FIG. 10 in accordance with one embodiment of the present disclosure;

FIG. 12 is a flow chart illustrating a user process for accessing the virtual waiting room of FIG. 10 in accordance with one embodiment of the present disclosure;

FIG. 13 is a flow chart illustrating a server process for providing access to the virtual waiting room as illustrated in FIG. 12 in accordance with one embodiment of the present disclosure; and

FIG. 14 is a schematic block diagram of one example of a system employed to register sick or injured persons for emergency medical services according to an embodiment of the present invention.

### DETAILED DESCRIPTION

Disclosed herein are various embodiments of systems and methods related to registering sick or injured persons for emergency medical services. Reference will now be made in detail to the description of the embodiments as illustrated in the drawings, wherein like reference numbers indicate like parts throughout the several views.

This disclosure describes systems and methods for preregistering sick or injured persons for emergency medical services over a network such as, but not limited to, the Internet. This process may allow patients to avoid waiting for an extended period of time in the waiting room at an emergency department or urgent care center before receiving treatment.

An estimated waiting time associated with a participating emergency medical facility, such as, but not limited to, a hospital or urgent care center, is used in the patient preregistration process. In some embodiments, among others, a triage nurse (or some other designated party) at the participating hospital or urgent care center periodically estimates the length of time it would take for a new non-urgent, non-emergency patient to be seen by the doctor or physician assistant. The evaluation of the estimated wait time may be based upon factors such as, but not limited to, the volume and acuity of patients already triaged, the number of patients waiting to be triaged, the status of patients presently being treated, the complaints of the patients in the hospital waiting room, ambulance traffic that may be en route, the number and proficiency of care providers (or other support staff) on duty or scheduled to come on duty, and the number of examination/treatment rooms in the facility.

In other embodiments, the estimated wait time may be determined from data in the facility's registration system. For example, the registration database for the facility may be automatically monitored to determine the current wait time. The determined wait time may be automatically transmitted to the web site to update the current wait time of the facility. The determined wait time may be transmitted on a regular scheduled basis or when the wait time changes by a predetermined amount.

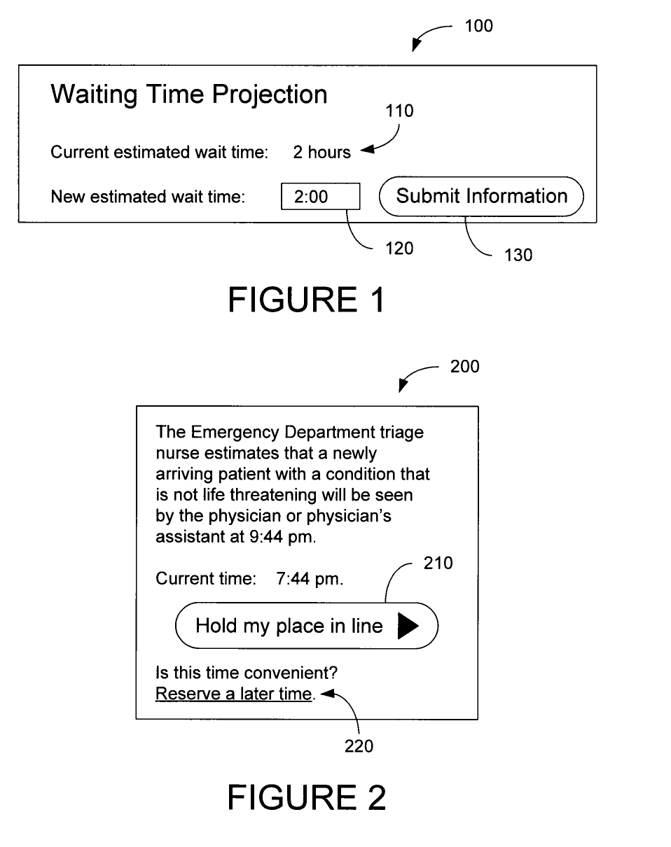

FIG. 1 is an illustration of a triage nurse waiting time entry form 100 in accordance with one embodiment of the present disclosure. The waiting time entry form 100 may display the value of the current estimated waiting time 110 of the participating hospital or urgent care center. Using the waiting time entry form of FIG. 1, the triage nurse enters the estimated wait time in data entry box 120 and submits the information for processing by selecting the confirm key 130. The triage nurse may continue to enter new estimates on a periodic basis (e.g., intervals of 10, 15, 20, and 30 minutes). In some embodiments, the triage nurse (or other designated party) may be prompted to update the estimated wait time. The submitted wait time is then used to update the current estimated waiting time 110.

In the embodiment shown in FIG. 1, the triage nurse enters the estimated wait time though a web page. In other embodiments, the estimated wait time may be entered by telephone or text messaging. In order to maintain the security of the submitted information, a login procedure utilizing a username and password may be used to gain access to a waiting time entry form on the web site. In some embodiments, the username corresponds to the participating facility, hospital, or urgent care center. In other embodiments, the username may correspond to an individual associated with the participating hospital or urgent care center. This allows for tracking and identification of the source of changes in the entered wait time.

The web pages may be hosted on a server which is accessed by a client. The client-server architecture may be in the form of a web server and/or a browser-client. The current and submitted wait times may be stored in a database. The database may reside on the same server as the web site. In other embodiments, the database may be on another local or remote server, which may be accessed by the web server.

When a sick or injured person wants to use the service, that person (or a responsible caregiver such as, but not limited to, a parent, relative, or custodian) accesses the registration web site. The web site provides for display of a list of available emergency medical facilities such as, but not limited to, hospitals or urgent care centers, and may include the current estimated wait time at each facility. The wait times that are displayed may be based upon the estimated wait time at the time the web site was accessed. In other embodiments, the displayed wait times may be updated on the basis of a refresh period or in response to the processing of submitted estimated wait times are processed.

In one non-limiting embodiment, every hospital using this service is provided for display on the registration web site. In other embodiments, hospitals are provided for display based upon geographic area. For example, a pull-down box may be used to select available geographic areas. Another embodiment may provide one hospital for display per web address or per web page. These are non-limiting embodiments and other possibilities could be used. If there is only one hospital or urgent care center available, the estimated waiting time for only the one facility may be displayed.

If there are several hospitals or urgent care centers available, the person accessing the registration web site may then select a facility that the patient will be taken to. The prospective patient (or responsible caregiver) may choose which hospital or urgent care center to register for from the displayed list. Once a facility is selected, a single waiting time display corresponding to the chosen facility may be provided. FIG. 2 is an illustration of a single hospital waiting time screen 200 in accordance with one embodiment of the present disclosure. In the embodiment of FIG. 2, the prospective patient may choose to hold his or her place in queue based upon the current wait time displayed by selecting option 210. The selected visitation time may be the arrival time, appointment time, admission time, time the patient may be sent to a treatment room, time a physician may see the patient, or other definable time. If a later time is preferred, the patient may submit that a request to reserve a later visitation time 220.

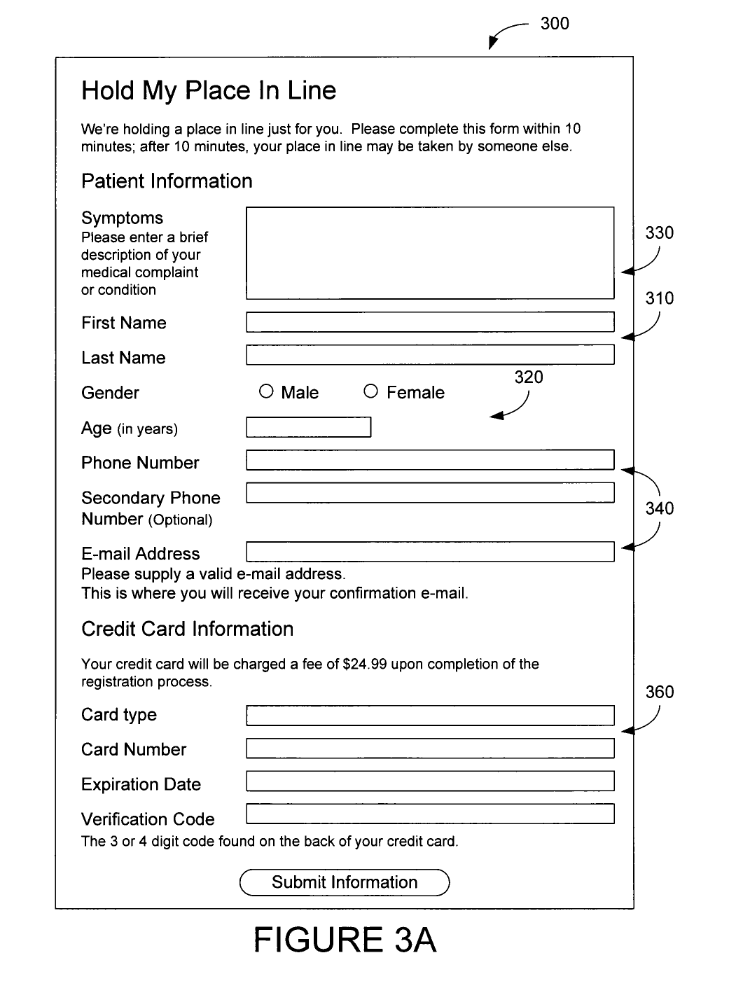

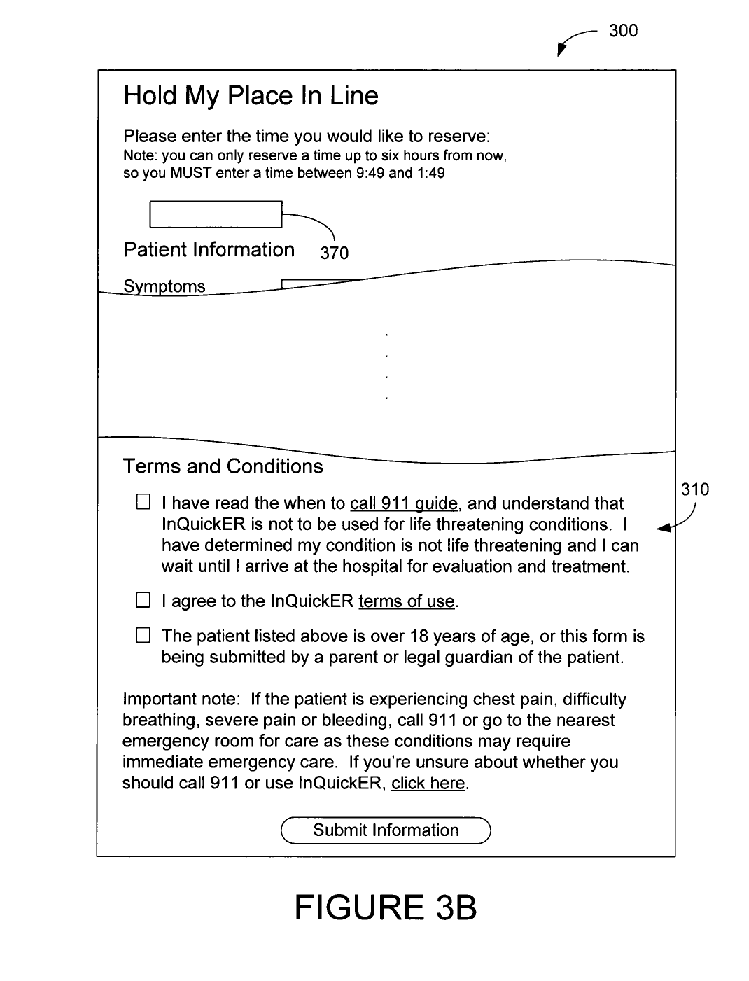

Upon selecting to reserve a time using option 210, a patient information form may be provided to gather additional patient information. FIG. 3A is an illustration of a patient information form 300 in accordance with one embodiment of the present disclosure. The prospective patient may then enter his or her name 310, some basic personal information (e.g., age, gender, etc.) 320, a brief description of the medical complaint (or symptoms) 330, and contact information (e.g., telephone numbers and e-mail address) 340 into the patient information form 300. Other information requested by the selected facility may also be included in the patient information form 300. Some embodiments, such as, but not limited to, the one illustrated in FIG. 3B, may also include one or more confirmations 350 of various terms and conditions by the person accessing the web site. The confirmation 350 may regard status information such as, but not limited to, confirmation that the patient's condition is not life threatening, confirmation that the patient is of legal age, and/or confirmation that the person accessing the web site is the legal guardian of the patient. Other embodiments may also provide for submission of credit card information 360. The credit card information may also be supplied to the selected hospital or urgent care center for registration, billing, or other purposes. In some embodiments, a confirmation of the reservation will be sent to the potential patient by e-mail.

In other embodiments, the prospective patient may select a more convenient or preferred time he or she would like to arrive at the hospital or urgent care center. By selecting option 220 of FIG. 2, patient information form 300 may allow the user to enter a preferred time as illustrated in FIG. 3B. In one embodiment, the prospective patient can choose any time between the current time plus the estimated wait length and an arbitrary outer limit. For example, if the triage nurse's estimated wait time is two hours and the hospital's outer limit for scheduling is twelve hours, then if the time is currently 7 am, the prospective patient may choose any time between 9 am and 7 pm on the current day. In the embodiment of FIG. 3B, the time limit is six hours and the preferred time is entered in box 370. In other embodiments, the prospective patient may schedule a window of time during which he or she will arrive at the facility. This may also be accomplished using a pull-down box with available times.

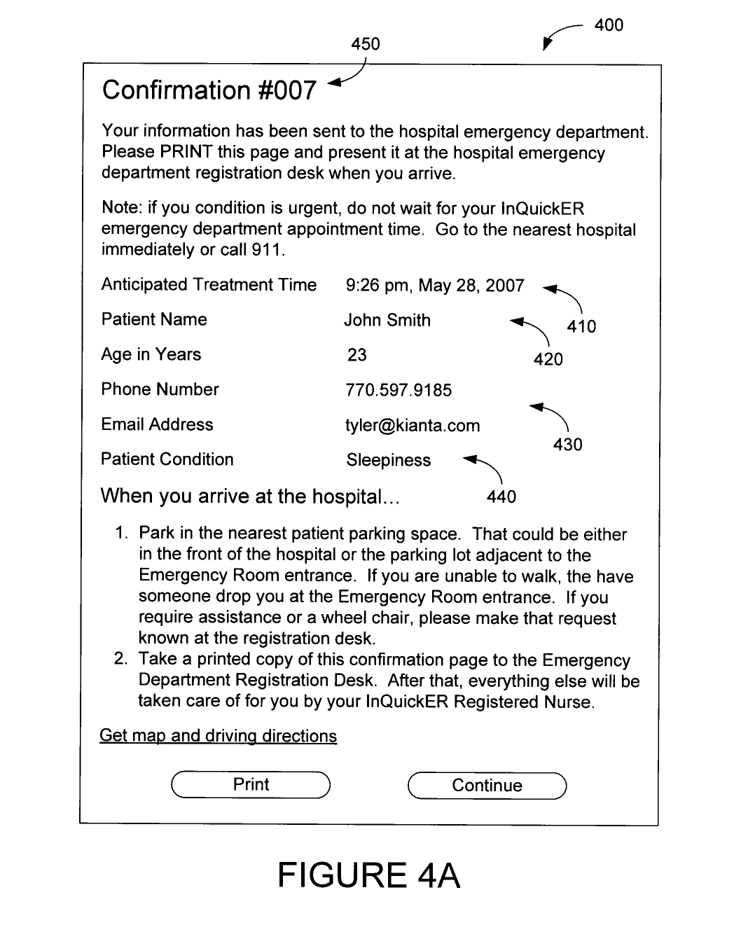

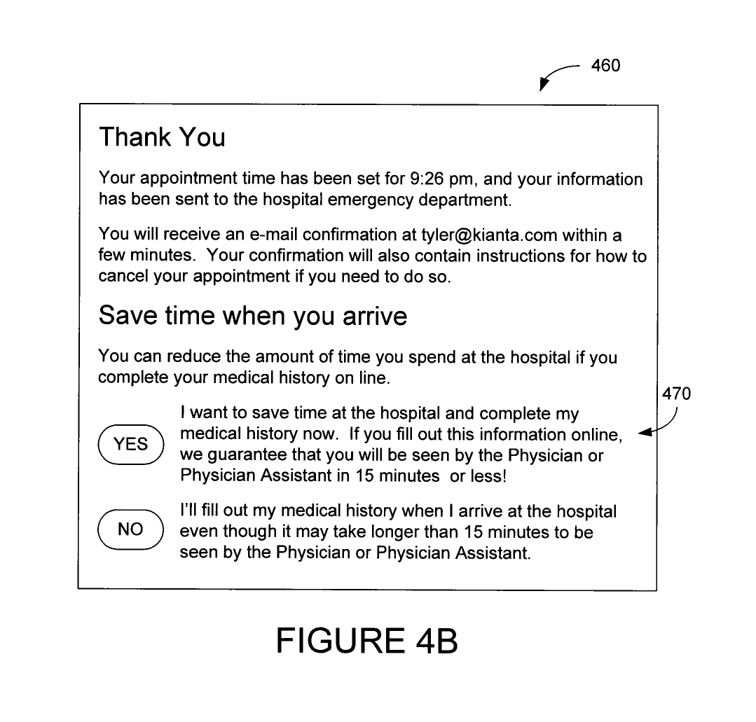

In response to the submission of a patient information form, confirmation of the registration is provided for display to the person accessing the web site. In other embodiments, the confirmation may be sent to the user using the provided e-mail address 340. FIG. 4A is an illustration of a patient confirmation screen 400 in accordance with one embodiment of the present disclosure. The patient confirmation screen 400 may include, but is not limited to, the scheduled or anticipated treatment time 410, patient name 420, contact information 430, and reported symptoms 440. The patient confirmation screen 400 may also provide access to directions to the selected facility and any other facility specific instructions which may aid in the treatment of the patient. The patient confirmation screen 400 may also include a confirmation number 450, which may be used during check in at the selected hospital or urgent care center. Additional hospital or urgent care center information may also be included such as, but not limited to, instructions, maps, or reminders to bring documentation.

In some embodiments, after reviewing and/or printing patient confirmation screen 400, an exiting confirmation screen 460 illustrated in FIG. 4B, may be provided for display to the prospective patient, which may give an option 470 for the submission of additional medical information. The prospective patient may submit a more extensive medical history, which may include, but is not limited to, current medications, long-term medical problems, and allergies. Providing the extended medical history may allow the patient to bypass some of the paperwork normally associated with an emergency room visit. In other embodiments, this may be requested through the patient confirmation screen 400.

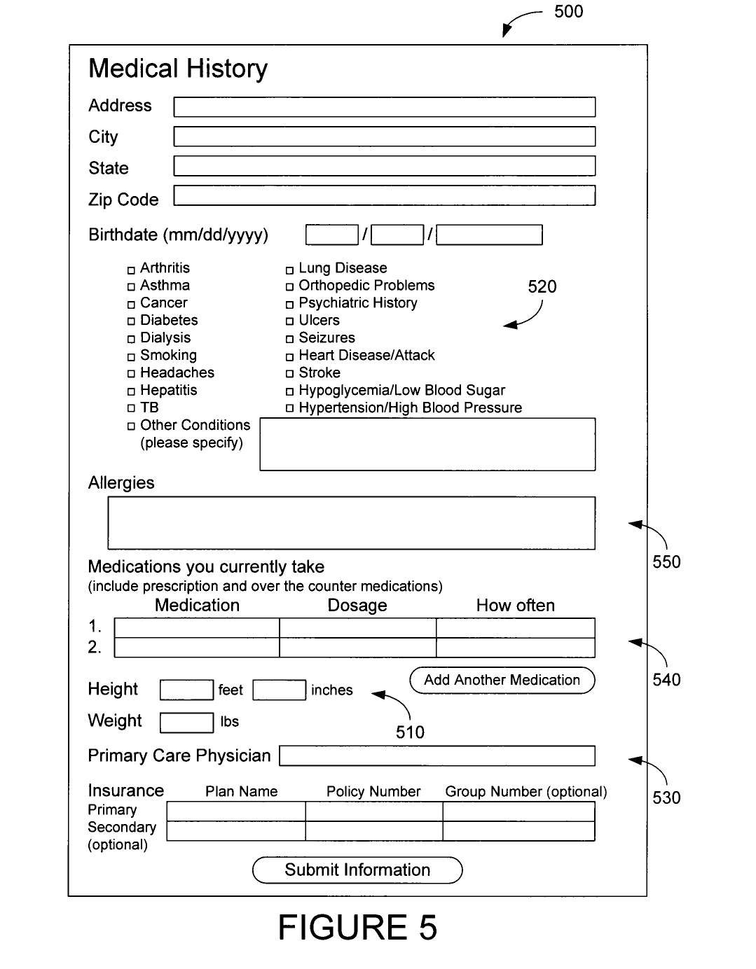

The prospective patient may provide the extended medical history by completing a medical history form through the web site. FIG. 5 is an illustration of a patient medical history form 500 in accordance with one embodiment of the present disclosure. The patient medical history form 500 may include, but is not limited to, physical information about the patient 510, known medical conditions 520, the patient's primary care physician 530, and any current medications 540 on known allergies 550. The patient medical history form 500 may also include medical insurance information or other information requested by the selected facility.

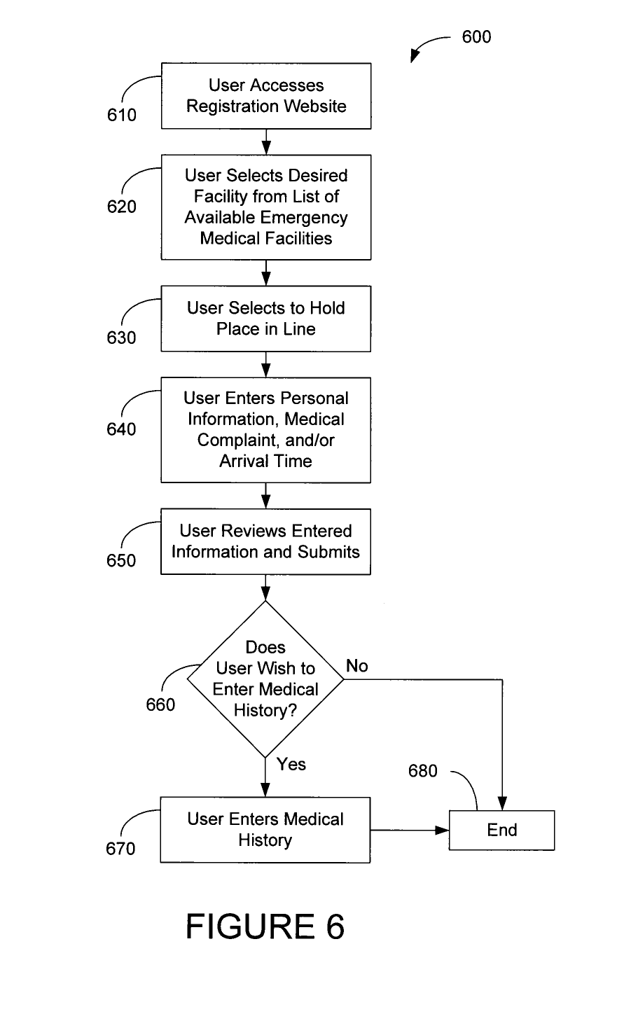

FIG. 6 is a flow chart 600 illustrating a user process for registering for emergency medical services using the forms of FIGS. 3-5 in accordance with one embodiment of the present disclosure. In one embodiment, a user (e.g., potential patient or responsible care giver) accesses a registration web site in block 610. A list of available emergency medical facilities (e.g., emergency rooms and urgent care centers) is displayed for the user's selection. The list may include one or more single facilities. The user selects the desired facility from the list in block 620. The current waiting time for the selected facility is provided to the user through a display such as, but not limited to, the waiting time screen 200 illustrated in FIG. 2. The user may then select to hold the current waiting time in block 630 or reserve a later time.

The user enters patient information (block 640) including, but not limited to, the patient's personal information, medical complaint and/or arrival time using a patient information form, such as, but not limited to, form 300 illustrated in FIGS. 3A and 3B. The user reviews the entered information and submits the form in block 650. A confirmation of the reservation (e.g., the form of FIG. 4A) may be provided to the user. In addition, the user may be provided with the opportunity to enter more detailed patient medical history (block 660). If the user chooses to submit additional medical information, the user may enter the information in block 670 using a medical history form such as, but not limited to, form 500 illustrated in FIG. 5. After submission of the medical history, the process ends (block 680). If the user chooses not to submit additional medical history information, the process ends at block 680.

In other embodiments, a user's medical history may be submitted to the server and stored for later access by the user. If a medical history has been stored, the user may select the stored medical history for submission without additional input. In alternative embodiments, the user may edit the stored medical history before submission.

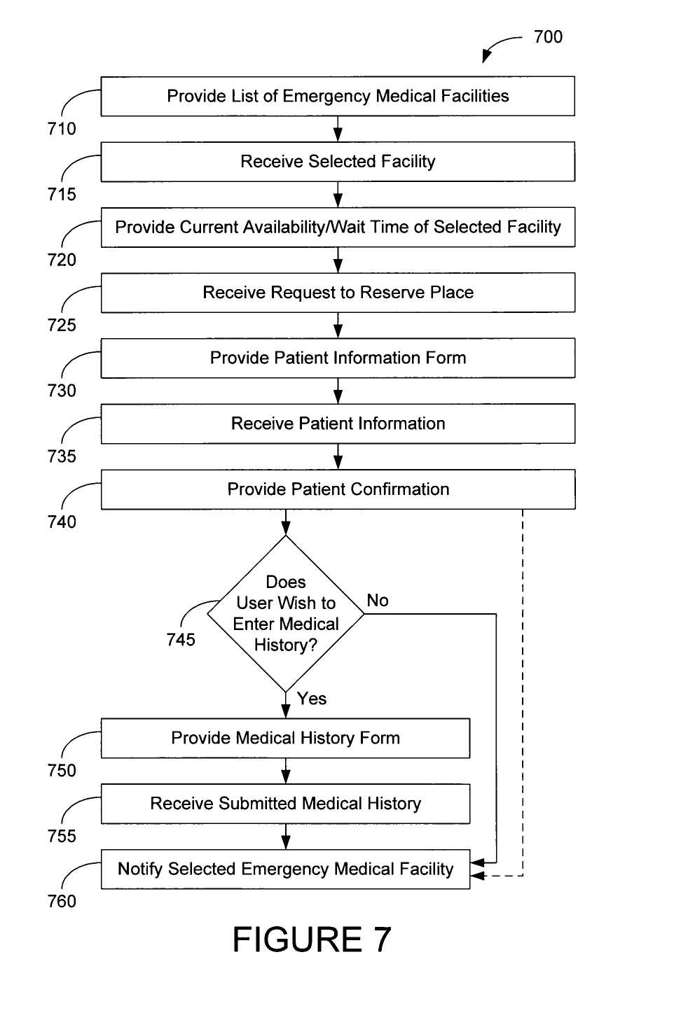

FIG. 7 is a flow chart 700 illustrating a server process for registering for emergency medical services as illustrated in FIG. 6 in accordance with one embodiment of the present disclosure. A server (e.g., a web server) may provide a list of emergency medical facilities for display to a user in block 710. In response to receiving an emergency medical facility selected from the list by a user (block 715), the web site may provide the current availability and/or wait time of the selected facility for display to the user in block 720. The user may then choose to hold a place in queue or exit the process. If the user chooses to reserve a place, the server receives the request (block 725) and provides a patient information form for display to the user. In one embodiment, among others, the patient information form 300 of FIG. 3A may be provided.

The server receives the patient information in block 735 when submitted by the user. The server may store the patient information as submitted or may reformat and/or encrypt the information before providing the information to the selected emergency medical facility. A patient confirmation may be provided to the user in block 740 to confirm receipt of the request. In one embodiment, the server may notify the selected facility of the user's request after block 740 (as indicated by the dashed line).

In other embodiments, the server may provide a prompt asking if the user wishes to enter additional medical history information (block 745). If the server receives an indication that the user does not wish to submit additional information, then the server notifies the selected facility of the user's request in block 760. If the user does wish to submit additional medical history information, a medical history form is provided for display in block 750. The server receives the submitted medical history information in block 755 and notifies the selected facility in block 760. The medical history information may be stored as submitted or may be reformatted and/or encrypted before providing the information to the selected emergency medical facility. The patient may be allowed access the stored medical history to allow for review and/or updating the information. Access may be granted using a username/password combination or other appropriate protection method or system.

Once the prospective patient has submitted his or her information, the triage nurse (or other designated party, such as, but not limited to, a physician or nurse practitioner) at the selected hospital is alerted of the new reservation request. The triage nurse or designated party may be informed of the request via pager, text messaging, cell phone, fax, e-mail, or other appropriate communication means. The nurse may then access the web site and view the prospective patient's details to ensure that he or she does not appear to have an urgent or emergent medical condition that may require immediate emergency care. In other embodiments, the server may provide an evaluation of the potential patient's condition and provide notification of the evaluation to the selected emergency medical facility, designated emergency medical personnel, the potential patient, or combinations thereof.

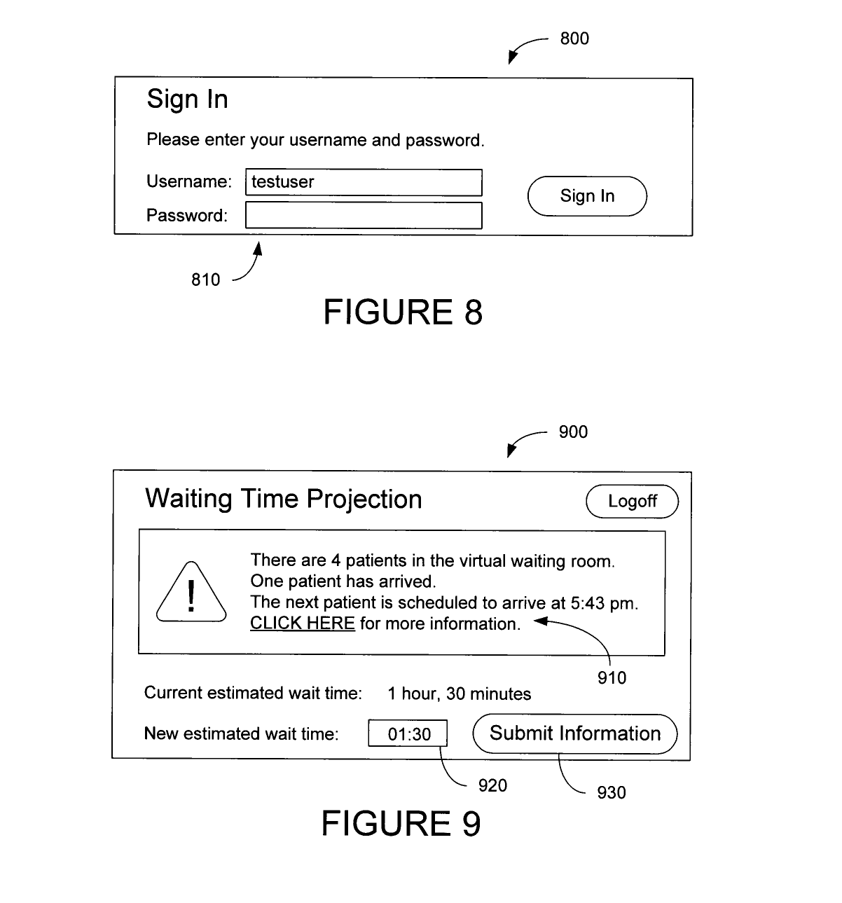

In some embodiments, emergency medical facility personnel may login for access to the web site. FIG. 8 is an illustration of an emergency medical facility login screen 800 in accordance with one embodiment of the present disclosure. The triage nurse (or other responsible party such as, but not limited to, a registration clerk) signs onto the employees' area of the web site by entering a username and password 810. In one embodiment, the username is associated with the emergency medical facility and may be used by all responsible parties to access web site information. In other embodiments, each responsible party designated by the facility has an individual username and password. This may also allow for controlling access to site information and monitoring of individuals who access to the patient information.

Once the nurse has signed in, he or she may be directed to a home page associated with the emergency medical facility. FIG. 9 is an illustration of a facility home page 900 including the triage nurse waiting time entry form information of FIG. 1 in accordance with one embodiment of the present disclosure. The nurse may determine from information displayed on the facility home page 900 the current status of patients who have registered online. The displayed information may include, but is not limited to, the number of patients that have registered online and the earliest expected arrival time. More information related to the registered patients may be examined by accessing a virtual waiting room through a link 910 on the facility home page 900.

In the embodiment of FIG. 9, the nurse or other designated individual may also change the estimated wait time by entering the new time in box 920 and submit 930 the new time for update from the facility home page 900. In other embodiments, a facility home page may only include a link to the triage nurse waiting time entry form 100 of FIG. 1. In one embodiment, editing the wait time initiates a communication from the web site to the patients who are currently in line regarding the change of status. This alert is sent by any available medium, non-limiting examples including email, fax, instant message or text message. In some embodiments, the nurse is allowed to edit the alert before transmission.

In one embodiment, the alert may be automatically set by the web site. The conditions under which the alert is automatically sent may be configurable by the facility. Exemplary configurations include, but are not limited to, increase in wait time of over 30 minutes, increase in wait time of over 25%, alert only sent to patients whose wait time is currently more than 30 minutes or currently more than one hour, alerts not sent unless expressly requested by the user when the wait time is edited, etc. In other embodiments, designated individuals of the facility may configure the conditions.

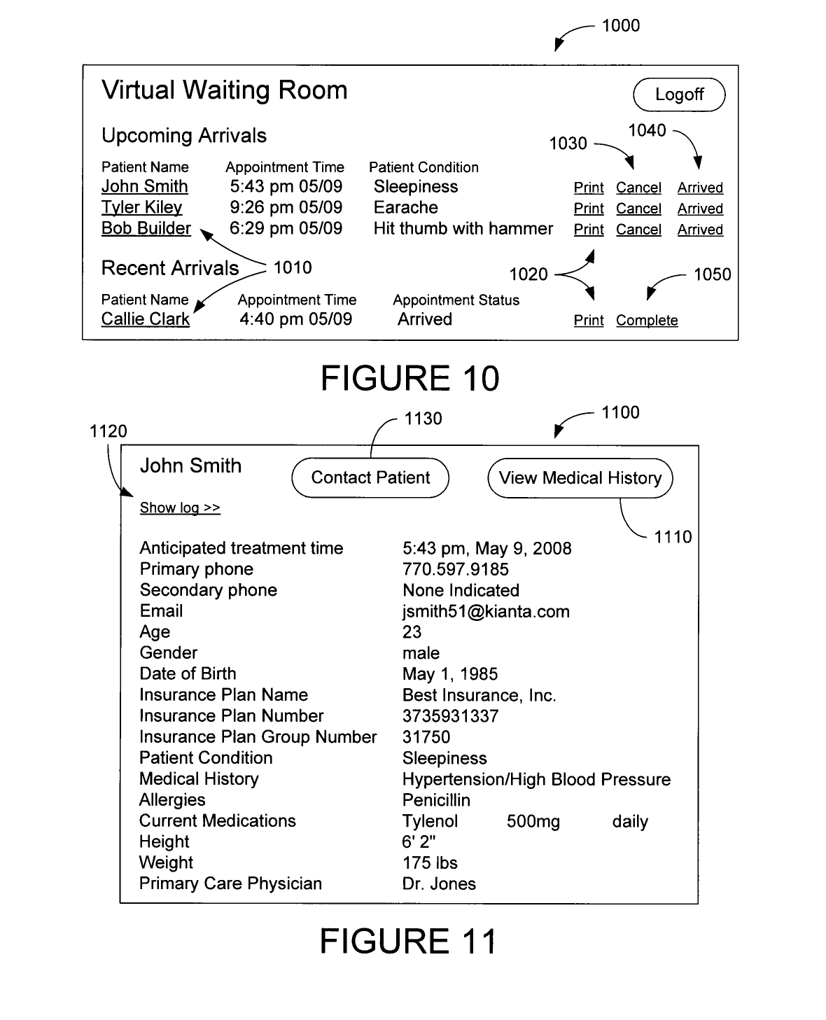

In the embodiment of FIG. 9, when a nurse selects the “CLICK HERE for more information” link 910, a virtual waiting room is provided for display including, but not limited to, a summary of recent patients and patients who will be arriving soon. FIG. 10 is an illustration of a virtual waiting room display 1000 which may be accessed from the facility home page 900 of FIG. 9 in accordance with one embodiment of the present disclosure. In the embodiment of FIG. 10, the patient's name, appointment time, and symptoms/condition are provided for display. Other embodiments may include different combinations of the information submitted by the patient. In addition, the patient listings may be divided between patients scheduled for arrival (upcoming arrivals) and patients who have checked in (recent arrivals). In other embodiments, the facility home page may include the virtual waiting room.

The virtual waiting room 1000 may also include various functions available to the person accessing the information. In the embodiment of FIG. 10, the nurse may view a summary of the details provided by patients when they registered online by selecting the patient name 1010. Other embodiments may use a separate button to access the patient information. The nurse may also print the patient information using link 1020.

FIG. 11 is an illustration of a patient information summary 1100 which may be viewed 1010 or printed 1020 from the virtual waiting room 1000 of FIG. 10 in accordance with one embodiment of the present disclosure. The summary 1100 may include all of the submitted patient information and medical history. In other embodiments, only a portion of the patient information submitted using the patient information form 300 (FIG. 3A) may be displayed. In the embodiment of FIG. 11, a complete medical history of the patient may be viewed by selection of button 1110. A log of the patient's medical information may also be accessed by selecting the show log option 1120. The log may include information such as, but not limited to, a record of chances to the information and who made the change, a record of who has viewed the information, and a record of previous appointments.

Upon accessing the submitted patient information, the triage nurse, physician, physician's assistant, or other appropriate personal evaluate the information to determine the prospective patient's condition. If the prospective patient's condition is not urgent or emergent, the nurse may take no action and await the arrival of the patient. If the prospective patient's condition appears to be urgent or emergent, the nurse may attempt to contact the prospective patient (using the information provided during the registration process, e.g. from the patient information form 300) and instruct him or her to proceed to the nearest hospital or urgent care center immediately. If the triage nurse needs additional information to evaluate the patient's condition, the nurse may contact the patient to obtain the information before the patient's arrival.

In some embodiments, this communication may be facilitated through the web site. This could be in the form of email, fax, instant message, text message, telephone call or any other available medium. In the embodiment of FIG. 11, the communication to the patient is initiated by the web site by selecting option 1130. By selecting option 1130, the web site may initiate contact between the patient and the facility using a method, which may be selected from a list of options or predetermined. While the communication may be initiated by web site, the nurse is able to determine the content of the communication. If the communication has a textual form, the web site creates a default message to be sent which the nurse has the opportunity to edit. If the communication is via telephone, the nurse is connected directly to the patient. In some embodiments, emergency response services may also notified and/or dispatched in response to the triage nurse request. In other embodiments, the nurse may contact the patient without web site assistance based upon the submitted patient information.

When the prospective patient arrives at the hospital or urgent care center at his or her selected appointment time, he or she checks in at the registration desk. The registration clerk (or other designated person) at the hospital retrieves the patient's information from the web site through the virtual waiting room associated with the facility. If the additional medical history has been submitted, the patient may be allowed to bypass the queue in the waiting room without having to wait and fill out any forms. The patient may proceed directly to the treatment area to be seen by a doctor.

The status of an appointment may also be changed from the virtual waiting room 1000. For example, in the embodiment of FIG. 10, the appointments of upcoming arrivals may be canceled 1030 or changed to arrived 1040 when the patient checks in. When a patient arrives at the hospital, the registration clerk, nurse, or other designated person can select the arrived option 1040 on the virtual waiting room screen 1000. In the embodiment of FIG. 10, that action moves the patient from the upcoming arrivals section of the virtual waiting room 1000 into the recent arrivals section. In other embodiments, the patient may be moved into an archive upon selecting the arrived option 1040. If a patient cancels his or her visit, the nurse selects the cancel option 1030, which removes the appointment from the virtual waiting room 1000. In some embodiments, the appointment information is moved into an archive. In other embodiments, the information may be completely deleted.

In addition, the appointment for recent arrivals may be completed 1050 when criteria specified by the emergency medical facility is met. The available options on virtual waiting room 1000 are not intended to be an exhaustive list. For example, other embodiments may include a link which allows the nurse to edit or change information entered by the patient, including, but not limited to, the arrival time. A change in this information could be configured to trigger an alert or facilitated communication to the potential patient as discussed above.

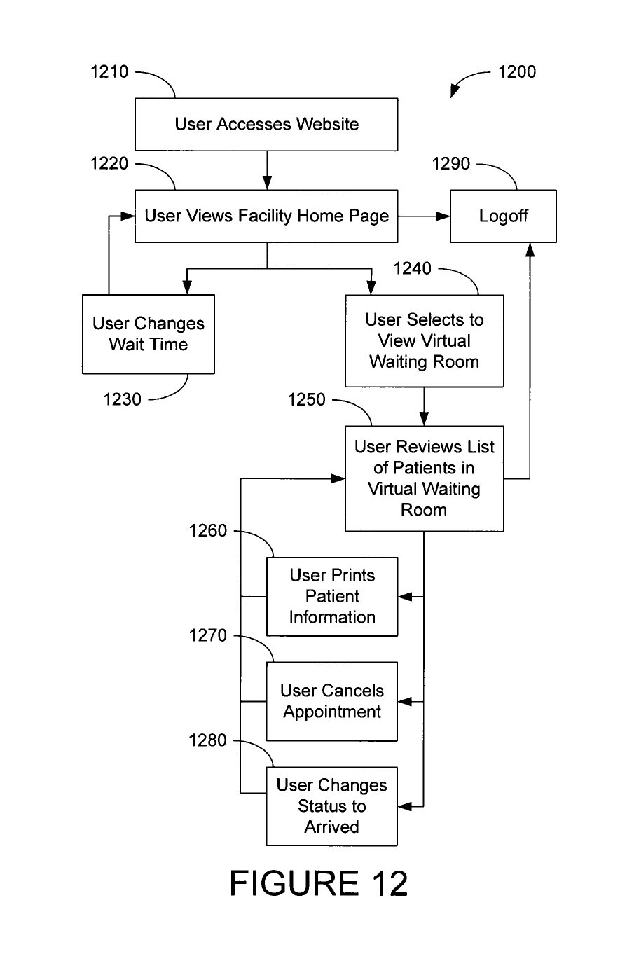

FIG. 12 is a flow chart 1200 illustrating a user process for accessing the virtual waiting room 1000 of FIG. 10 in accordance with one embodiment of the present disclosure. In the embodiment of FIG. 12, a user such as, but not limited to, a triage nurse accesses the web site in block 1210 using a username and password as illustrated in FIG. 8. Other embodiments may utilize other verification techniques. The user may then view a facility home page (block 1220) such as, but not limited to, the facility home page 900 illustrated in FIG. 9. From the facility home page, the user may select to change the current wait time of the emergency medical facility in block 1230, to view the virtual waiting room associated with the facility in block 1240, or to logoff in block 1290.

If the user chooses to view the virtual waiting room in block 1240, the user may review a list of patients in the virtual waiting room such as those illustrated in FIG. 10. Patient information and/or medical history of each patient may be examined. For some or all of the listed appointments, the user may also choose to print the patient information in block 1260, cancel the appointment in block 1270, and change the status to arrived in block 1280 when the patient checks in. After selecting each action, the user may be returned to the virtual waiting room for further review (block 1250). In some embodiments, the user may also logoff (block 1290) from the server.

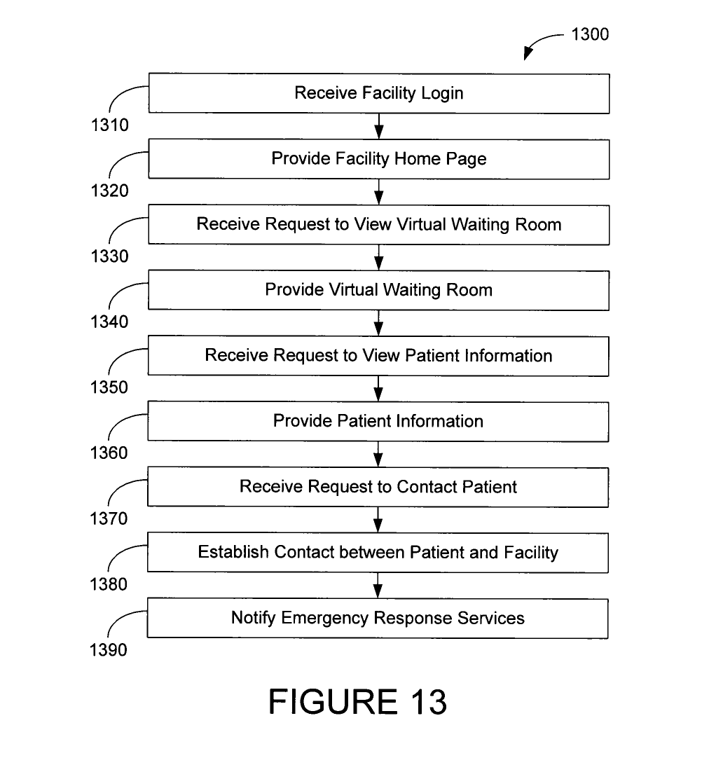

FIG. 13 is a flow chart 1300 illustrating a server process for providing access to the virtual waiting room 1000 as illustrated in FIG. 12 in accordance with one embodiment of the present disclosure. In block 1310, the web site (or web server) receives a facility login from a user. Upon confirming that the login is valid, the facility home page is provided for display to the user (e.g., the triage nurse) in block 1320. In one embodiment, the facility home page 900 of FIG. 9 is provided. The facility home page may be the same for all users associated with a facility or may be customized for each user. The web site may then receive a variety of requests from the user such as, but not limited to, updating the current estimated wait time for the facility and viewing a virtual waiting room associated with the facility. In the embodiment of FIG. 13, the web site receives a request to view the virtual waiting room in block 1330 and provides the information for display in block 1340.

The user may then review the patient listing and select an option associated with one of the listed patients. In the embodiment of FIG. 13, the user chooses to view the medical information of one of the listed patients. The web site service receives the request in block 1350 and provides the selected patient information in block 1360. The user may then evaluate the provided information to determine the patient's condition. If the user determines that immediate attention is needed by the patient, the user may request that the web site contact the potential patient. When the request is received (block 1370), the web site may establish contact between the patient and the facility, user, and/or other designated emergency medical personnel in block 1380. In some embodiments, emergency response services may also be notified of the situation in block 1390.

In other embodiments, the web site may determine the condition of the patient and establish contact between the patient and the facility, user, and/or other designated emergency medical personnel in block 1380 in response to the determination. Emergency response services may also be notified of the situation (block 1390) responsive to the determination.

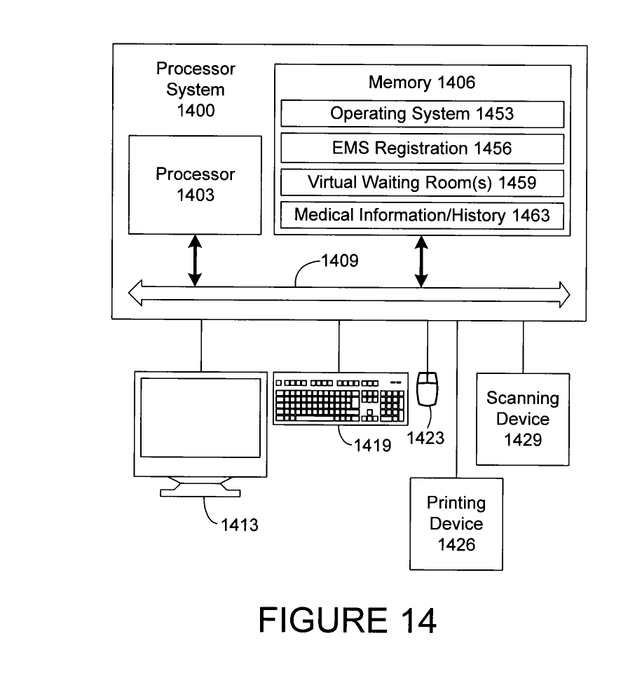

Referring next to FIG. 14, shown is one example of a system that performs various functions using methods related to registering sick or injured persons for emergency medical services according to the various embodiments as set forth above. As shown, a processor system 1400 is provided that includes a processor 1403 and a memory 1406, both of which are coupled to a local interface 1409. The local interface 1409 may be, for example, a data bus with an accompanying control/address bus as can be appreciated by those with ordinary skill in the art. The processor system 1400 may comprise, for example, a computer system such as a server, desktop computer, laptop, personal digital assistant, or other system with like capability.

Coupled to the processor system 1400 are various peripheral devices such as, for example, a display device 1413, a keyboard 1419, and a mouse 1423. In addition, other peripheral devices that allow for the capture of various patterns may be coupled to the processor system 1400 such as, for example, a printing device 1426, or a scanning device 1429.

Stored in the memory 1406 and executed by the processor 1403 are various components that provide various functionalities according to the various embodiments of the present invention. In the example embodiment shown, stored in the memory 1406 is an operating system 1453 and an emergency medical service (EMS) registration system 1456. In addition, stored in the memory 1406 are various virtual waiting rooms 1459 and various patient medical information and/or histories 1463. The patient medical information 1463 may be associated with corresponding ones of the various virtual waiting rooms 1459. The virtual waiting rooms 1459 and the patient medical information/history 1463 may be stored in a database to be accessed by the other systems as needed. The virtual waiting rooms 1459 may comprise listings and options such as those illustrated in FIG. 10, or others as can be appreciated.

The emergency medical service registration system 1456 is executed by the processor 1403 in order to preregister sick or injured persons for emergency medical services as described above. A number of software components are stored in the memory 1406 and are executable by the processor 143. In this respect, the term “executable” means a program file that is in a form that can ultimately be run by the processor 1403. Examples of executable programs may be, for example, a compiled program that can be translated into machine code in a format that can be loaded into a random access portion of the memory 1406 and run by the processor 1403, or source code that may be expressed in proper format such as object code that is capable of being loaded into a of random access portion of the memory 1406 and executed by the processor 1403, etc. An executable program may be stored in any portion or component of the memory 1406 including, for example, random access memory, read-only memory, a hard drive, compact disk (CD), floppy disk, or other memory components.

The memory 1406 is defined herein as both volatile and nonvolatile memory and data storage components. Volatile components are those that do not retain data values upon loss of power. Nonvolatile components are those that retain data upon a loss of power. Thus, the memory 1406 may comprise, for example, random access memory (RAM), read-only memory (ROM), hard disk drives, floppy disks accessed via an associated floppy disk drive, compact discs accessed via a compact disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

The processor 1403 may represent multiple processors and the memory 1406 may represent multiple memories that operate in parallel. In such a case, the local interface 1409 may be an appropriate network that facilitates communication between any two of the multiple processors, between any processor and any one of the memories, or between any two of the memories etc. The processor 1403 may be of electrical, optical, or molecular construction, or of some other construction as can be appreciated by those with ordinary skill in the art.

The operating system 1453 is executed to control the allocation and usage of hardware resources such as the memory, processing time and peripheral devices in the processor system 1400. In this manner, the operating system 1453 serves as the foundation on which applications depend as is generally known by those with ordinary skill in the art.

Although the emergency medical service (EMS) registration system 1456 is described as being embodied in software or code executed by general purpose hardware as discussed above, as an alternative the same may also be embodied in dedicated hardware or a combination of software/general purpose hardware and dedicated hardware. If embodied in dedicated hardware, the emergency medical service registration system 1456 can be implemented as a circuit or state machine that employs any one of or a combination of a number of technologies. These technologies may include, but are not limited to, discrete logic circuits having logic gates for implementing various logic functions upon an application of one or more data signals, application specific integrated circuits having appropriate logic gates, programmable gate arrays (PGA), field programmable gate arrays (FPGA), or other components, etc. Such technologies are generally well known by those skilled in the art and, consequently, are not described in detail herein.

The flow charts of FIGS. 6-7 and 12-13 show the architecture, functionality, and operation of an implementation of the emergency medical service registration system 1456. If embodied in software, each block may represent a module, segment, or portion of code that comprises program instructions to implement the specified logical function(s). The program instructions may be embodied in the form of source code that comprises human-readable statements written in a programming language or machine code that comprises numerical instructions recognizable by a suitable execution system such as a processor in a computer system or other system. The machine code may be converted from the source code, etc. If embodied in hardware, each block may represent a circuit or a number of interconnected circuits to implement the specified logical function(s).

Although flow charts of FIGS. 6-7 and 12-13 show a specific order of execution, it is understood that the order of execution may differ from that which is depicted. For example, the order of execution of two or more blocks may be scrambled relative to the order shown. Also, two or more blocks shown in succession in FIGS. 6-7 and 12-13 may be executed concurrently or with partial concurrence. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present invention.

Also, where the emergency medical service registration system 1456 may comprise software or code, each can be embodied in any computer-readable medium for use by or in connection with an instruction execution system such as, for example, a processor in a computer system or other system. In this sense, the logic may comprise, for example, statements including instructions and declarations that can be fetched from the computer-readable medium and executed by the instruction execution system. In the context of the present invention, a “computer-readable medium” can be any medium that can contain, store, or maintain the emergency medical service registration system 1456 for use by or in connection with the instruction execution system. The computer readable medium can comprise any one of many physical media such as, for example, electronic, magnetic, optical, electromagnetic, infrared, or semiconductor media. More specific examples of a suitable computer-readable medium would include, but are not limited to, magnetic tapes, magnetic floppy diskettes, magnetic hard drives, or compact discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Another embodiment, among others, includes a method, comprising: providing a current wait time for an emergency medical facility; receiving a request to reserve a visitation time for the emergency medical facility, the visitation time after the current wait time; requesting patient information; and notifying the emergency medical facility of the reserved visitation time. The method may further comprise: receiving patient information; and providing a confirmation of the received patient information and reserved visitation time. The method may further comprise requesting patient medical history information. The method may further comprise providing the received patient information to the emergency medical facility. The emergency medical facility may be selected from a list of available emergency medical facilities. The method may further comprise: providing the list of available emergency medical facilities; and receiving the emergency medical facility selected from the list. The list of available medical facilities may be based upon geographic location. Requesting patient information may comprise providing patient information form for entry of patient information. Receiving patient information may comprise receiving information entered into a patient information form. Requesting patient medical history information may comprise providing a patient medical history form for entry of patient medical history information. The visitation time may be an arrival time.

Another embodiment, among others, includes a method, comprising: receiving a request to view a virtual waiting room associated with an emergency medical facility; and providing the virtual waiting room including a list of patients who have a reserved visitation time for the emergency medical facility. The method may further comprise: receiving a request to view patient information associated with a patient listed in the virtual waiting room; and providing the requested patient information for display. The method may further comprise: receiving a request to contact the patient listed in the virtual waiting room; and establishing contact between the patient listed in the virtual waiting room and the emergency medical facility. The contact established with the emergency medical facility may be with a triage nurse. The contact may be established through an e-mail. The method may further comprise notifying an emergency response service. The method may further comprise: receiving a username and a corresponding password; determining the emergency medical facility associated with the username; and providing a facility home page for display, the facility home page associated with the emergency medical facility.

Another embodiment, among others, includes a method, comprising: receiving a username and a corresponding password; determining an emergency medical facility associated with the username; receiving an estimated waiting time associated with the emergency medical facility; updating a current waiting time associated with the emergency medical facility; and providing notification of the updated current waiting time to a patient having a reserved visitation time for the emergency medical facility. The method may further comprise providing notification of the updated current waiting time to the emergency medical facility.

It should be emphasized that the above-described embodiments of the present invention are merely possible examples of implementations, merely set forth for a clear understanding of the principles of the invention. Many variations and modifications may be made to the above-described embodiment(s) of the invention without departing substantially from the spirit and principles of the invention. All such modifications and variations are intended to be included herein within the scope of this disclosure and the present invention and protected by the following claims.

Therefore, at least the following is claimed:

1. A method, comprising:
providing a current wait time for an emergency medical facility;
receiving a request to reserve a visitation time for the emergency medical facility, the visitation time after the current wait time;
requesting patient information; and
notifying the emergency medical facility of the reserved visitation time.

2. The method of claim 1, further comprising:
receiving patient information; and
providing a confirmation of the received patient information and reserved visitation time.

3. The method of claim 2, further comprising requesting patient medical history information.

4. The method of claim 2, further comprising providing the received patient information to the emergency medical facility.

5. The method of claim 1, wherein the emergency medical facility is selected from a list of available emergency medical facilities.

6. The method of claim 5, further comprising:
providing the list of available emergency medical facilities; and
receiving the emergency medical facility selected from the list.

7. The method of claim 6, wherein the list of available medical facilities is based upon geographic location.

8. The method of claim 1, wherein requesting patient information comprises
providing patient information form for entry of the patient information.

9. The method of claim 2, wherein receiving patient information comprises receiving information entered into a patient information form.

10. The method of claim 3, wherein requesting patient medical history information comprises providing a patient medical history form for entry of the patient medical history information.

11. The method of claim 1, wherein the visitation time is an arrival time.

12. A method, comprising:
receiving a request to view a virtual waiting room associated with an emergency medical facility; and
providing the virtual waiting room including a list of patients who have a reserved visitation time for the emergency medical facility.

13. The method of claim 12, further comprising:
receiving a request to view patient information associated with a patient listed in the virtual waiting room; and
providing the requested patient information for display.

14. The method of claim 13, further comprising:
receiving a request to contact the patient listed in the virtual waiting room; and
establishing contact between the patient listed in the virtual waiting room and the emergency medical facility.

15. The method of claim 14, wherein the contact established with the emergency medical facility is with a triage nurse.

16. The method of claim 14, wherein the contact is established through an e-mail.

17. The method of claim 14, further comprising notifying an emergency response service.

18. The method of claim 12, further comprising:
receiving a username and a corresponding password;
determining the emergency medical facility associated with the username; and
providing a facility home page for display, the facility home page associated with the emergency medical facility.

19. A method, comprising:
receiving a username and a corresponding password;
determining an emergency medical facility associated with the username;
receiving an estimated waiting time associated with the emergency medical facility;
updating a current waiting time associated with the emergency medical facility; and
providing notification of the updated current waiting time to a patient having a reserved visitation time for the emergency medical facility.

20. The method of claim 19, further comprising providing notification of the updated current waiting time to the emergency medical facility.
